(19) United States
(12) Patent Application Publication
(10) Pub. No.: US 2007/0032850 A1
(43) Pub. Date: Feb. 8, 2007

(54) Separable sheath and method for insertion of a medical device into a bodily vessel using a separable sheath

(76) Inventors: Carlos Ruiz, New York, NY (US); Gerald Dorros, Wilson, WY (US)

Correspondence Address: KENYON & KENYON LLP, ONE BROADWAY, NEW YORK, NY, 10004, US

(21) Appl. No.: 11/435,814

(22) Filed: May 16, 2006

Related U.S. Application Data
(63) Continuation-in-part of application No. 11/303,758, filed on Dec. 15, 2005.
(60) Provisional application No. 60/593,173, filed on Dec. 16, 2004.

(51) Int. Cl. A61F 2/06

(52) U.S. Cl. 623/1.11

(57) ABSTRACT

A separable insertion sheath is for inserting a medical device into a patient. The insertion sheath includes releasably connectable ends. A medical device loaded into the insertion sheath is deployed by using an elongate member connected to the insertion sheath to shift the proximal and distal sections away from each other to expose the medical device.

### CROSS-REFERENCE TO RELATED APPLICATIONS

This application is a continuation-in-part of U.S. patent application Ser. No. 11/303,758, entitled “A Separable Sheath and Method for Insertion of a Medical Device into a Bodily Vessel Using a Separable Sheath,” filed in the United States Patent and Trademark Office on Dec. 15, 2005, which is expressly incorporated herein in its entirety by reference thereto.

### INCORPORATION BY REFERENCE

U.S. Provisional Patent Application No. 60/593,173, filed on Dec. 16, 2004 and entitled “Prosthetic Valve,” is expressly incorporated herein in its entirety by reference thereto. Each of U.S. patent application Ser. No. 11/303,761, entitled “A Heart Valve and Method for Insertion of the Heart Valve Into a Bodily Vessel,” filed in the United States Patent and Trademark Office on Dec. 15, 2005, and U.S. patent application Ser. No. 11/303,758, entitled “A Separable Sheath and Method for Insertion of a Medical Device into a Bodily Vessel Using a Separable Sheath,” filed in the United States Patent and Trademark Office on Dec. 15, 2005, is also expressly incorporated herein in its entirety by reference thereto.

### FIELD OF THE INVENTION

The present invention relates to a separable sheath and a method for insertion of a medical device into a bodily vessel using a separable sheath.

### BACKGROUND INFORMATION

Various methods exist for transcatheter implantation of a medical device into a bodily vessel of a patient. For example, angioplasty procedures may involve implantation of an expandable stent using a balloon catheter. The balloon catheter is typically advanced into the vasculature of a patient through a sheath. The sheath is at least partially withdrawn to expose the stent, which is expanded by inflating a balloon of the balloon catheter onto which the stent is disposed and in a similar manner with self-expanding stents that are currently deployed by withdrawing a sheath and exposing the device. Valves, such as heart valves, can also be implanted transcatheter into a bodily vessel, for example, to replace native valves exhibiting abnormal anatomy and/or function as a result of congenital or acquired disease. Similar to the stents, expandable valves have been implanted using balloon catheters and self-expandable stents with mounted bioprosthesis or mechanical prosthesis.

Insertion of a medical device, such as a stent or valve, requires precise positioning and handling. Blood flow created by the beating of the heart and the tortuous nature of many bodily vessels increases the difficulty of such insertion. Therefore, there is believed to be a need for a medical device insertion device and method offering enhanced control and consistency.

### SUMMARY

A method according to an example embodiment of the present invention for implanting a medical device, such as a stent or valve, into a patient, includes inserting a separable or splittable sheath into the patient and pulling proximal and distal portions of the sheath away from each other so as to expose the medical device, which is at least partially disposed within an outside surface of at least one of the proximal and distal portions of the sheath. The medical device may be disposed within each of the proximal and distal portions of the sheath.

When inserting a self-expandable stent, for example, shifting of the proximal portion of the sheath away from the distal portion of the sheath allows a proximal end of the stent to expand and shifting of the distal portion of the sheath allows a distal end of the stent to expand. The proximal and distal portions of the sheath may also be pulled away from each other simultaneously allowing each end of the stent to expand simultaneously.

Releasably connectable ends of the proximal and distal portions of the sheath may be disconnected prior to pulling the proximal and distal portions away from each other.

Ends of the proximal and distal portions may be connected by a threaded connection. Disconnection of the ends may be accomplished by rotating the proximal and distal portions about a longitudinal axis of the sheath relative to each other.

Ends of the proximal and distal portions may be connected by a latch, and disconnection of the proximal and distal portion ends may be accomplished by disengaging the latch.

The latch may be pivotally connected to or integral with one of the proximal and distal portions, and the other of the proximal and distal portions may include a recess configured to receive a portion of the latch. The sheath may include a line extending along a length of one of the proximal and distal portions connected to the latch configured to allow the latch to be pivoted to disconnect the proximal and distal portions. The line may be slidable relative to the sheath and may be configured such that pulling of the line pivots the latch out of the recess so as to disconnect the proximal and distal portions.

The sheath may include a servo or motor configured to pivot the latch between the locked and unlocked positions. The line may be configured to transmit an electric control signal to the servo or motor to connect and/or disconnect ends of the proximal and distal portions of the sheath.

Ends of the proximal and distal portions may be connected by a magnetic force. For example, an end of at least one of the proximal and distal portions may include a magnet, e.g., an electro-magnet, configured to generate a magnetic field, and an end of the other of the proximal and distal portions may include a magnetically-attractable member, a permanent magnet, an electromagnet, etc., which is attracted to the magnetic field. Interruption of the magnetic field eliminates the magnetic force between the proximal and distal portions of the sheath and, therefore, effectively disconnects these portions.

Rather than including two separate portions that are releasably connected end-to-end, the sheath may include a single unit, which may be split into proximal and distal portions, for example, circumferentially, by pulling opposite ends of the sheath away from each other. The sheath may also be split by twisting the proximal and distal portions relative to each other.

The splittable sheath may include a weakened section or frangible section at a predetermined location along the length of the sheath to provide that the splitting of the sheath occurs a desired predetermined location on the sheath. Pulling the proximal and distal ends of the sheath away from each other at a predetermined pulling force or twisting the ends of the sheath relative to each other at a predetermined twisting force may cause failure at the weakened or frangible section thus splitting the sheath into proximal and distal portions. The wall of the sheath may have a reduced thickness or may be cut at the weakened section so as to facilitate splitting of the sheath.

The medical device may include any type of device that may be inserted via transcatheter deployment such as a stent, an endovascular graft, a valve, etc. The medical device may also include any of the devices described in U.S. patent application Ser. No. 11/303,761, entitled “A Heart Valve and Method for Insertion of the Heart Valve Into a Bodily Vessel,”filed in the United States Patent and Trademark Office on Dec. 15, 2005, which is expressly incorporated herein in its entirety by reference thereto. For example, the medical device may include a valve having separate first and second expandable sections. One of the expandable sections may be disposed or contained within one of the proximal and distal portions or on one side of the weakened section, and the other expandable section may be disposed or contained within the other of the proximal and distal portions or on another side of the weakened section.

The first and second expandable sections may be spaced apart and connected by struts. The struts may span the connection point or weakened section between the two expandable sections.

The first expandable section may be arranged as a valve, and the second expandable section may be configured to anchor the medical device in the patient.

The sheath may be inserted into the patient over a guidewire. The guidewire may be inserted through the femoral vein, inferior vena cava (IVC), right atrium (RA), left atrium (LA), left ventricle (LV), ascending and descending aorta (AO), abdominal aorta, and iliac artery, and may be exteriorized through the femoral artery.

The sheath may be positioned in the patient such that a distal end of the proximal portion and a proximal end of the distal portion of the sheath are adjacent to a deployment site for the medical device. For example, the deployment site may be in the aorta of the patient.

The medical device may be advanced into position in the sheath connected to or mounted on an insertion device, such as a balloon catheter. The medical device may be preloaded into the sheath prior to insertion of the sheath or may be advanced, for example, mounted on a balloon catheter, into an already inserted sheath.

The medical device may be arranged as a valve and may include a valve portion and anchor portion connected to the valve portion by one or more connectors. The valve portion and the anchor portion may be configured to be delivered into the bodily vessel in a low profile and to be expanded to a larger profile, and the anchor portion may be adapted to anchor the valve in place in the bodily vessel.

The anchor portion may be mechanically expandable (such as by a balloon inflation, a wrench, electrically, magnetically, etc.), self-expandable, and/or may be made from a shape-memory material, and may be constructed from an absorbable or non-absorbable material. The connector may include a strut extending along substantially an entire length of the valve portion, either longitudinally and/or perpendicularly in a circumferential manner at the level of the valve.

The valve portion may be substantially tubular and may include a plurality of flaps configured to allow fluid to pass therethrough in only one direction.

The valve portion may be made from biological materials, such as (a) small intestine sub-mucosa, (b) large tubular vascular structure, (c) pericardial tissue, (d) fascia lata, or (e) nano-synthesized material, such as stretchable Nitinol, etc. The valve portion may also be made from other biocompatible materials, such as ePTFE, silk, Elast-Eon™, etc.

The valve portion may be made of an invaginated tube, and an inner wall of the invaginated tube may be incised in at least two locations to form the flaps or leaflets, which permit unidirectional blood flow. The valve portion may be stentless. Alternatively, the valve portion may include a stent to maintain its expanded position.

The anchor portion may include a stent and may be tapered toward the valve portion, for example, in a cylindrical or truncated conical form.

The connector may have a C-shaped terminal end that is proximal to the anchor to support the radial expansion of the tissue valve.

The connector may include a T-shaped retainer securing the tubular tissue of the external portion of the invaginated tube to each connector.

The T-shaped retainer may be disposed within a slot in the connector, and the valve portion may be arranged between each T-shaped retainer and connector.

The valve portion may be created and secured to the connectors utilizing one or more of, for example, glue, rivets, suture, staples, etc.

The connector may be constructed as part of the anchor device or may be attached to the anchor, for example, utilizing one or more of a chemical or physical adherence technique, suture, staples, rivets, etc. A portion of the connector in contact with the valve portion may be ribbed and/or may include bores. The connector may be of sufficient length to allow the anchoring portion to fully expand while the valve portion remains in a low profile state.

A valve for placement in a bodily vessel includes: a stentless valve portion and an anchor portion situated end-to-end with the valve portion. Alternatively, the valve portion may include a stent to maintain its expanded position. Both expanded components may be attached so as to form a cylindrical or ovoid structure, with the anchor portion being self-expanding so as to attach to the walls of the bodily vessel. The stentless valve may be directly adherent end-to-end to the anchor portion which thereby obviates the necessity for a connector, such as a strut attachment, between the anchor and the valve. During insertion, the valve portion may be contained within one of the proximal and distal portions of the sheath and the anchor portion may be contained within the other of the proximal and distal portions of the sheath.

A method for insertion of a valve includes: a) placing a guide wire through the femoral vein, inferior vena cava (IVC), right atrium (RA), left atrium (LA), left ventricle (LV), ascending and descending aorta (AO), abdominal aorta, iliac artery, and exteriorizing the guide wire through the femoral artery; b) passing an insertion sheath, e.g., a sheath splittable (capable of being divided, for example, circumferentially) into proximal and distal portions or a sheath having releasably connectable proximal and distal portions, over the guide wire such that a distal end of the sheath is exteriorized through the femoral artery; c) passing an insertion device, such as a balloon catheter, over the guide wire and through the sheath such that a valve device of the present invention mounted to the insertion device is in deployment position near the anatomical location of the native aortic valve, wherein, when a balloon catheter is used, an anchoring portion of the valve device is disposed over a distal balloon and a stentless valve portion is disposed over a proximal balloon of the balloon catheter, and wherein a proximal end of the distal portion of the sheath is disposed over the anchoring portion and a distal end of the proximal portion of the sheath is disposed over the valve portion of the valve device; d) at least partially withdrawing the proximal portion of the sheath from the patient via the femoral vein so as to expose the valve portion; e) inflating the proximal balloon of the balloon catheter so as to expand the valve portion of the valve device, the valve device now being fully deployed; f) deflating the proximal balloon of the balloon catheter; g) at least partially withdrawing a distal portion of the sheath through the femoral artery cannulation site (which may optionally include a sheath system) so as to expose the anchoring portion; h) inflating the distal balloon so as to expand the anchoring portion; i) deflating the distal balloon; and j) removing the balloon catheter, guide wire and sheath from the patient.

The distal balloon of the balloon catheter may be deflated before or after deflation of the proximal balloon.

The guide wire may be placed in step (a) using any suitable guide wire insertion method. For example, the guide wire may be placed using the techniques of transseptal catheterization, which involves floating a balloon catheter in the direction of blood flow through the left atrium, left ventricle, and into the aorta, which is then retrogradely snared. In a version of the conventional technique, the. insertion sheath is advanced into the left atrium (LA) using its own dilator. The dilator is pulled out and the balloon catheter is then advanced through the sheath and exteriorized in the left atrium (LA). Once in the left atrium (LA), a balloon on the balloon catheter is inflated and floated out of the left ventricle (LV) through the aortic valve into the descending aorta, across the aortic arch and into the descending aorta. The wire is then be passed through the floating balloon catheter and exteriorized in the descending aorta. Once the balloon catheter is exteriorized, a retrograde advanced snare device is advanced retrogradely through the femoral artery and snares the tip of the wire and exteriorizes the wire out through the femoral artery, thereby completing the loop through the heart from the femoral vein to the femoral artery. See, for example, Babic et al., Percutaneous Mitral Valvuloplasty: Retrograde, Transarterial Double-Balloon Technique Utilizing the Transseptal Approach, Catheterization and Cardiovascular Diagnosis, 14:229-237 (1988), which is expressly incorporated herein in its entirety by reference thereto. The transseptal sheath may be sufficiently large to provide passage of the guidewire and splittable or releasably connectable two-part sheath through it into the ascending aorta.

The anchoring portion may be self-expandable. When a balloon catheter is used, it need only have a single balloon for inflation of the valve portion of the valve device. Alternatively, the distal balloon may be used in conjunction with a self-expandable anchoring portion, for example, to provide complete expansion of the anchoring portion.

A valve system includes a medical device, such as a valve or stent, and an insertion sheath sized for insertion of the medical device into a bodily vessel. The insertion sheath may either (i) include proximal and distal portions that are releasably connectable to each other or (ii) may be configured to split into the proximal and distal portions at a predetermined location upon pulling of the proximal and distal portions away from each other at a predetermined pulling force or twisting the proximal and distal portions relative to each other at a predetermined twisting force. The medical device may be configured to be delivered into the bodily vessel through or in the insertion sheath and positionable within the sheath such that pulling of the proximal and distal portions away from each other exposes the medical device. The proximal and distal portions of the insertion sheath may be releasably connected, for example, by a threaded connection, a magnetic connection, a latch, etc.

The sheath may include a sealable chamber which is configured to be sealed when the proximal and distal portion are connected and opened when the proximal and distal portions are separated.

The insertion sheath may include one or more elongate members, such as a stylet or threaded connecting member, attached to it. The elongate member may be used to shift the proximal and distal portions relative to each other. The stylet may be slidingly disposed in a first one of the proximal and distal portions of the sheath and secured to a second one of the proximal and distal portions of the sheath. A mechanism may be used to shift the stylet and second one of the proximal and distal portions of the sheath relative to the first one of the proximal and distal portions of the sheath. The mechanism may include, for example, one or more powered wheels configured to roll on the stylet to shift it along the longitudinal axis of the sheath. Further, the mechanism may include gears configured to engage teeth on the elongate member, e.g., the mechanism may be arranged as a rack-and-pinion mechanism.

As an alternative to or in combination with the stylet, a threaded connecting member may be threaded in a first one of the proximal portion and distal portion of the sheath and connected to a second one of the proximal portion and distal portion. The threaded connecting member may rotate relative to the second one of the proximal portion and distal portion. A mechanism may be used to rotate the threaded connector relative to the first one of the proximal and distal portions of the sheath to shift the proximal and distal portions relative to each other. The mechanism may include, for example, a rotating head connected to the threaded connecting member and configured to rotate the threaded connecting member.

The mechanisms for rotating and/or shifting the elongate member may be computer controlled to assure precise control of the relative positioning of the proximal and distal portions of the sheath. To enhance precision and control of the relative positioning of the proximal and distal portions, the mechanism for rotating and/or shifting may include one or more encoders or sensors for determining relative movement between the proximal and distal portions.

The sheath may also include a visualization device, such as an ultrasound device, e.g., an ultrasound catheter, wire, camera or transducer, which provides for visualization of the vessel or organ structure and allows for exact placement of an implantable device carried by the sheath. The visualization device may be connected to a display device such as a monitor.

Exemplary embodiments of the present invention are described in more detail below with reference to the appended Figures.

### BRIEF DESCRIPTION OF THE DRAWINGS

FIG. 1a is a cross-sectional view of an insertion sheath system according to an exemplary embodiment of the present invention inserted over a guidewire into the heart and vasculature of a patient, illustrated in cross-section.

FIG. 1b illustrates the insertion sheath system illustrated in Figure la with proximal and distal portions of the sheath disconnected and shifted away from each other exposing a stent illustrated in side view.

FIG. 1c is a side view of a fully deployed stent in the aorta.

FIG. 2a illustrates the insertion sheath system illustrated in FIG. 1a inserted over a balloon catheter illustrated in an inflated state expanding the stent.

FIG. 2b illustrates the insertion sheath system illustrated in FIG. 2a with a balloon of the balloon catheter in a deflated state.

FIG. 3 is a side view of valve according to an exemplary embodiment of the present invention.

FIG. 4a is a side view of an insertion sheath according to an exemplary embodiment of the present invention inserted into the heart and vasculature of a patient, illustrated in cross-section.

FIG. 4b illustrates the sheath illustrated in FIG. 4a with a proximal portion partially retracted revealing a proximal balloon of a balloon catheter extending through the sheath, which has been inflated to expand a valve portion of the valve included inside the insertion sheath.

FIG. 4c illustrates the balloon catheter illustrated in FIG. 4b with the proximal balloon deflated and with a distal portion of the insertion sheath removed revealing the distal balloon of the balloon catheter, which has been inflated to expand an anchor portion of the valve.

FIG. 4d is a side view of the valve illustrated in FIG. 4c fully implanted into the aorta.

FIG. 5A is a perspective view of the valve portion of the valve illustrated in FIG. 3 in a closed state illustrated without an optional cloth covering and the connectors and with a portion of the valve wall removed.

FIG. 5B is a perspective view of the valve portion of the valve illustrated in FIG. 5A in a closed state.

FIG. 5C is a perspective view of the valve portion of the valve illustrated in FIG. 3 in an open state illustrated without an optional cloth covering and the connectors.

FIG. 5D is a perspective view of the valve portion of the valve illustrated in FIG. 5C with a portion of the valve wall removed.

FIG. 6 is a cross-sectional view of the valve taken along line 6-6 in FIG. 3 illustrating a cross-sectional shape of connectors.

FIG. 7 is a cross-sectional view of the valve taken along line 7-7 in FIG. 3 illustrating a connection between a connector and the valve portion.

FIG. 8A is a side view of a valve according to an exemplary embodiment of the present invention.

FIG. 8B is a side view of a valve according to an exemplary embodiment of the present invention.

FIG. 9 is a longitudinal cross-sectional view of a threaded connection connecting proximal and distal portions of the insertion sheath.

FIG. 10 is a longitudinal cross-sectional view of a threaded connection of the insertion sheath.

FIG. 11 is a perspective view of an insertion sheath including a magnetic connector system connecting proximal and distal portions of the insertion sheath.

FIG. 12 is a longitudinal cross-sectional view of a latch connection connecting proximal and distal portions of the insertion sheath.

FIG. 13 is a longitudinal cross-sectional view of a latch connection connecting proximal and distal portions of the insertion sheath.

FIG. 14 is a longitudinal cross-sectional view of an insertion sheath including stylet.

FIG. 15 is a longitudinal cross-sectional view of an insertion sheath including a threaded connector.

### DETAILED DESCRIPTION

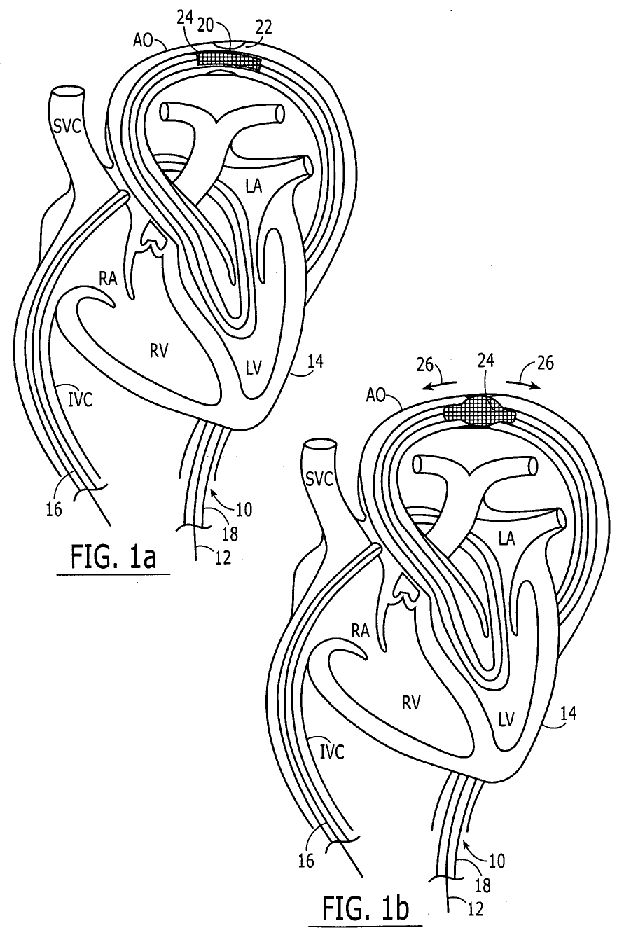

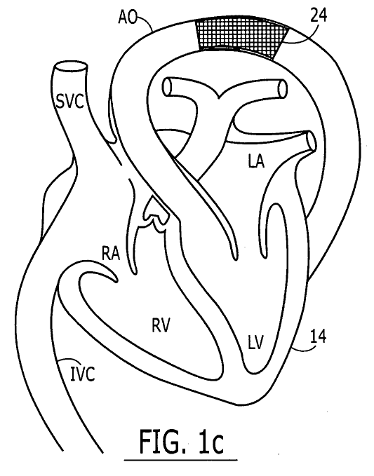

FIG. 1a illustrates an insertion sheath 10 inserted into a patient over a guidewire 12. The patient's heart 14 and vasculature are illustrates in cross-section. The guidewire 12 may be placed using any suitable guide wire insertion method. For example, the guide wire 12 may be placed using the techniques of transseptal catheterization, which includes floating a balloon catheter in the direction of blood flow through the left atrium (LA), left ventricle (LV), and into the aorta (AO), which is then retrogradely snared. In a version of a conventional technique, the insertion sheath is advanced into the left atrium (LA) using its own dilator. The dilator is pulled out and the balloon catheter is then advanced through the sheath and exteriorized in the left atrium (LA). Once in the left atrium (LA), a balloon on the balloon catheter is inflated and floated out of the left ventricle (LV) through the aortic valve into the descending aorta, across the aortic arch and into the descending aorta. The wire is then passed through the floating balloon catheter and exteriorized in the descending aorta. Once the balloon catheter is exteriorized, a retrograde advanced snare device is advanced retrogradely through the femoral artery and snares the tip of the wire and exteriorizes the wire out through the femoral artery, thereby completing the loop through the heart from the femoral vein to the femoral artery. See, for example, Babic et al., Percutaneous Mitral Valvuloplasty: Retrograde, Transarterial Double-Balloon Technique Utilizing the Transseptal Approach, Catheterization and Cardiovascular Diagnosis, 14:229-237 (1988), which is expressly incorporated herein in its entirety by reference thereto. The transseptal sheath may be sufficiently large to enable passage of the guidewire 12 and splittable or releasably connectable two-part sheath 10 through it into the ascending aorta.

The sheath 10 may be implanted using a retrograde approach, e.g., approaching the aortic valve from the descending aorta, or using an antegrade approach, e.g., approaching the aortic valve from the left ventricle after performing, for example, a transseptal puncture.

The sheath 10 may be separable into a proximal portion 16 and a distal portion 18. The sheath 10 may be positioned, for example, such that contact point 20, i.e., the location where the connecting ends of the proximal and distal portions 16, 18 come together, is located in a narrowed portion 22 of the aorta (AO). X-ray supervision, injection of X-ray traceable liquids, intravascular or intracardiac ultrasound, ultrasonic measuring, etc., may be used to assist in positioning the sheath 10. A medical device, such as an expandable stent 24, may be arranged in a low profile state within a proximal end of the distal portion 18 of the sheath 10 and within a distal end of the proximal portion 16 of the sheath 10. Shifting of the proximal and distal portions 16, 18 of the sheath 10 away from each other in the direction of arrows 26 illustrated in FIG. 1b exposes the stent 24 and allows it to expand and enlarge the narrowed portion 22 of the aorta (AO). FIG. 1b illustrates the proximal and distal portions 16, 18 of the sheath 10 partially withdrawn exposing a middle portion of the stent 24. FIG. 1c illustrates the stent 24 fully expanded and successfully enlarging the previously narrowed lumen in the aorta. The sheath 10 and guide wire 12 have been removed from the patient.

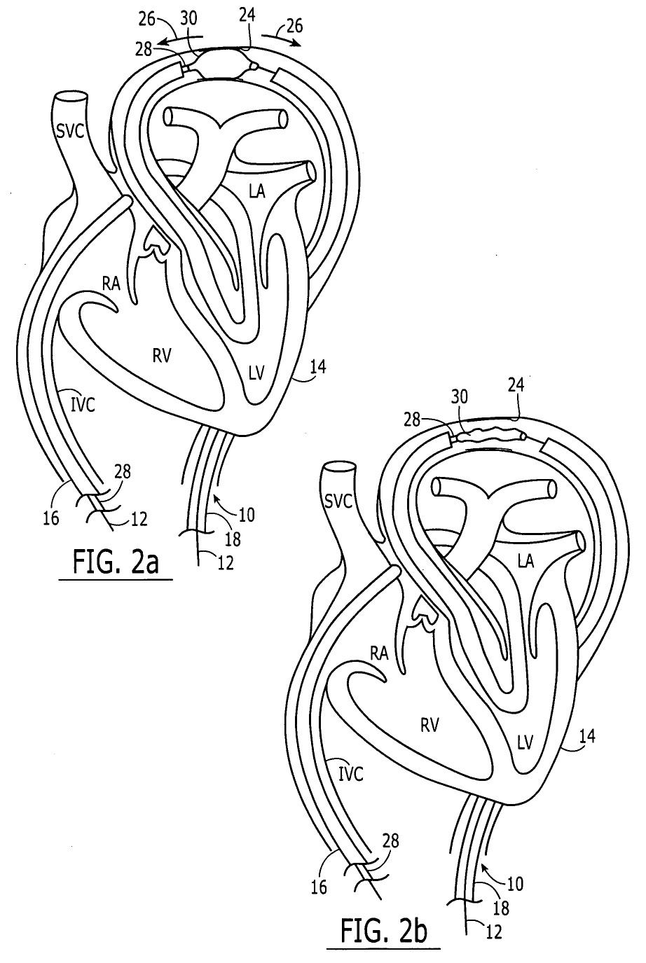

The stent 24 may be preloaded into the sheath 10 prior to insertion of the sheath 10 into the patient and may be advanced with the sheath 10 into the patient. The stent 24 may also be connected to or mounted on an insertion device, such as a balloon catheter 28, which may be advanced into the sheath 10 prior to or after insertion of the sheath 10 into the patient, or may be expanded using a retractable self expanding stent or any other retractable expandable device capable of expanding the stent 24. The balloon catheter 28 with the stent 24 disposed thereon may be positioned in the sheath 10 such that a portion of the stent 24 is arranged within each of the proximal and distal portions 16, 18 of the sheath 10. Shifting of the proximal and distal portions 16, 18 of the sheath 10 away from each other in the direction of arrows 26 exposes the stent 24 and balloon 30 of the balloon catheter. The sheath 10 may extend beyond an end of the balloon catheter 30 and may be tapered to a size which allows free passage and movement over the guide wire 12. As illustrated in FIG. 2a, inflation of balloon 30 expands the stent 24 to enlarge the lumen in the narrowed portion 22 of the aorta (AO). The balloon 30 may also be used in conjunction with a self-expandable stent to provide complete expansion of the stent. FIG. 2b illustrates the state after the balloon 30 has been deflated leaving the expanded stent 24 in place in the aorta (AO). The sheath 10, guide wire 12 and balloon catheter 28 are removed from the patient leaving the stent 24 in place.

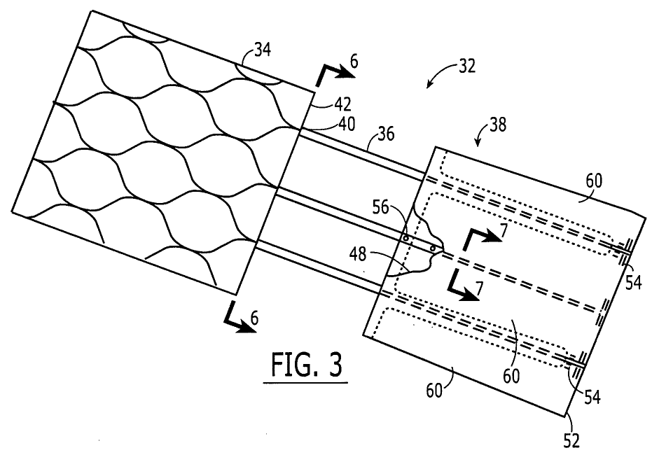

The insertion method may also be used to insert a valve 32, such as a heart valve illustrated in FIG. 3. Valve 32 may include an anchor portion 34, connectors 36 and a valve portion 38 spaced a distance away from anchor portion 34.

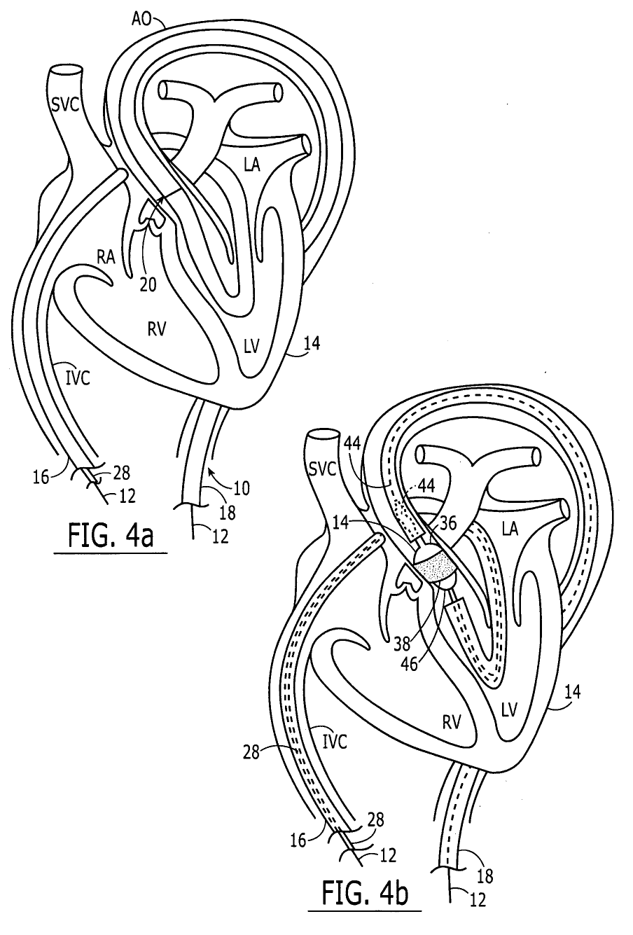

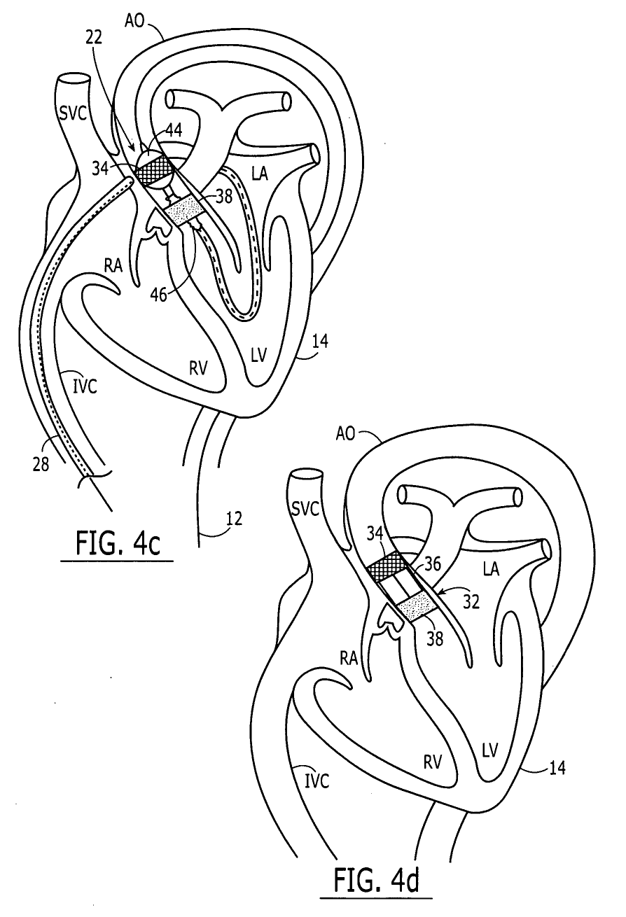

As illustrated in FIG. 4a, the sheath 10 may be positioned such that contact point 20, i.e., the location where the connecting ends of the proximal and distal portions 16, 18 of sheath 10 come together, is located in the patient at the desired deployment site for the valve 32, for example, near the anatomical location of the native aortic valve. Further, X-ray supervision, injection of X-ray traceable liquids, intravascular or intracardiac ultrasound, ultrasonic measuring, etc., may also be used to assist in positioning the sheath 10. An insertion device, such as balloon catheter 28, as illustrated in FIGS. 4b and 4c, may be advanced, for example, over the guidewire 12 through the sheath 10 such that distal balloon 44 is located on one side of the contact point 20 and proximal balloon 46 is positioned on an opposite side of the contact point 20. The valve portion 38 of the valve 32 may be disposed over the proximal balloon 46 and the anchoring portion 12 may be disposed over the distal balloon 44. Valve portion 38 may be disposed in the proximal portion 16 of the sheath 10 prior to deployment and is illustrated in dashed lines in FIG. 4b. As an alternative to placement of the sheath 10 first and then advancing the balloon catheter 28 into position within the sheath 10, the balloon catheter 28 may be disposed within the sheath 10 and advanced into position, for example, over guidewire 12 together with the already inserted sheath 10.

The proximal portion 16 of the sheath 10 may be at least partially withdrawn from the patient, for example, through the venous system,. thus exposing the valve portion 38 of the valve 32. The proximal balloon 46 may then be inflated so as to expand the valve portion 38, as illustrated in FIG. 4b. At this point, proximal balloon 46 may be shifted if the position of the valve portion 38 requires adjusting. The connectors 36 may be of sufficient length to allow the valve portion 38 to fully expand while the anchor portion 34 remains in a low profile state within sheath 10. The proximal balloon 46 may be deflated, which provides for the valve portion 38 to be fully expanded and functional. The distal portion 18 of the sheath 10 may be shifted toward the femoral artery cannulation site, thus exposing the anchoring portion 34 of the valve 32, as illustrated in FIG. 4c. The distal balloon 44 may be inflated so as to fully expand the anchoring portion 34 in the aorta (AO). Anchoring portion 34 may also be self-expandable, in which case the distal balloon 44 may not be necessary but may still be used to provide complete expansion of the anchoring portion 34. Thus, if a self-expandable anchoring portion 34 is used, the balloon catheter 28 may have a single balloon. The balloon catheter 28 may be removed from the patient, for example, through the venous system. FIG. 4d illustrates the implanted valve 32 after the sheath 10, balloon catheter 28 and guidewire 12 have been completely removed from the patient.

Rather than entirely removing the proximal portion 16 of the sheath 10 to expose the valve portion 38, the proximal portion 16 may be partially removed (enough to completely expose the valve portion 38) and then may be removed together with the balloon catheter 28 after valve 32 is fully implanted.

The proximal balloon 46 may be inflated before the distal balloon 44 to allow for positional adjustments of the valve 32 prior to anchoring. Alternatively, proximal balloon 46 and distal balloon 44 may be inflated simultaneously or distal balloon 44 may be inflated before proximal balloon 46.

Balloon catheter 28 may have only a single balloon. Valve portion 38 may not need to be expanded by a balloon because blood flow in the aorta (AO) may cause valve portion 38 to fully expand. Anchor portion 34 may be self-expandable and, therefore, may also not need to be expanded by a balloon.

The valve portion 38 and anchor portion 34 maybe self-expandable and/or expandable using a retractable device. For example, the valve portion 38 and anchor portion 34 may be expanded using a balloon on, for example, a balloon catheter, or expanded using a retractable self expanding stent or any other suitable retractable expandable device capable of expanding the valve portion and/or anchor portion.

Connectors 36 of valve 32 may extend along the commissural lines of the valve portion 38 a sufficient length to provide a strong connection with the valve portion 38. The connectors 36 may also be connected to the valve portion 38 at different points along its circumference. Connectors 36 are connected on a distal end 40 to a proximal end of the anchor portion 34. Connectors 36 may extend at least partially along the length of the anchor portion 34. Connectors 36 may be connected to anchor portion 34, for example, by welding, suturing, gluing, clipping, rivets, etc. Connectors 36 may also be integral with anchor portion 34.

The valve portion 38 may be covered by a cloth 48 made from, for example, DACRON®, but also may be used without any such covering. The portion of the connectors 36 connected to the valve portion 38 may be arranged between the cloth 48 and the valve portion 38, as illustrated, or may be connected to an inner or outer surface of the anchor portion 34. The valve portion 38 may be tapered toward the anchor portion 34. The connectors 36 may include ribs, such as T-shaped ribs 54, illustrated in dashed lines, to provide additional support to a proximal end 52 of the valve portion 38 and also to further secure connection of the connectors 36 to the valve portion 38. Furthermore, the connectors 36 may include bores 56 for passage of sutures to connect to the valve portion 38. The connectors 36 may be manufactured by injection molding, machining, using nano-synthesized metals, etc.

The valve portion 38 may be supported solely via its connection to the connectors 16 and, in effect, may be suspended by the anchor portion 34. Valve portion 38 may or may not have an additional stent disposed within or over it, which may adversely affect the performance of the valve 32. That is, valve portion 38 may be stentless. Alternatively, valve portion 38 may include a stent to maintain its expanded position.

Valve portion 38 may be made from biological materials, such as (i) small intestine sub-mucosa (SIS), (b) large tubular vascular structure, e.g., IVC, superior vena cava (SVC), aorta (AO), etc., (c) pericardial tissue, (d) fascia lata, (e) nano-synthesized material, such as Nitinol, (f) or other biocompatible materials such as urethane, polyurethane, polyethylene terephthalate (PET), polytetrafluoroethylene (PTFE), expanded PTFE, silk, Rayon, DACRON@, etc. The valve portion 38 may also be made from a suitable plastic, such as Elast-Eon™, a metal, metal alloy, etc.

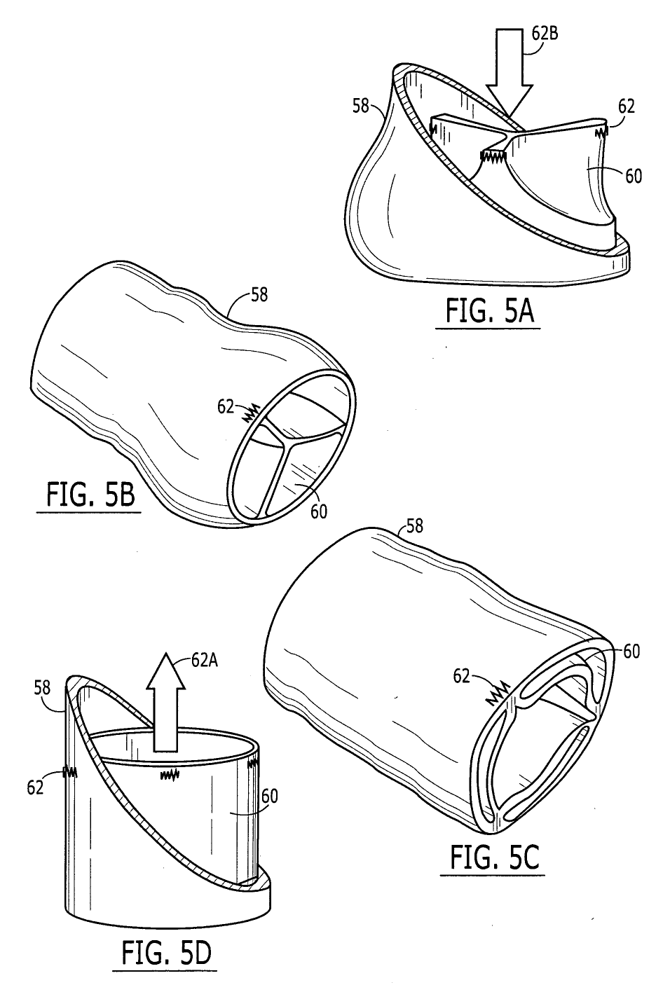

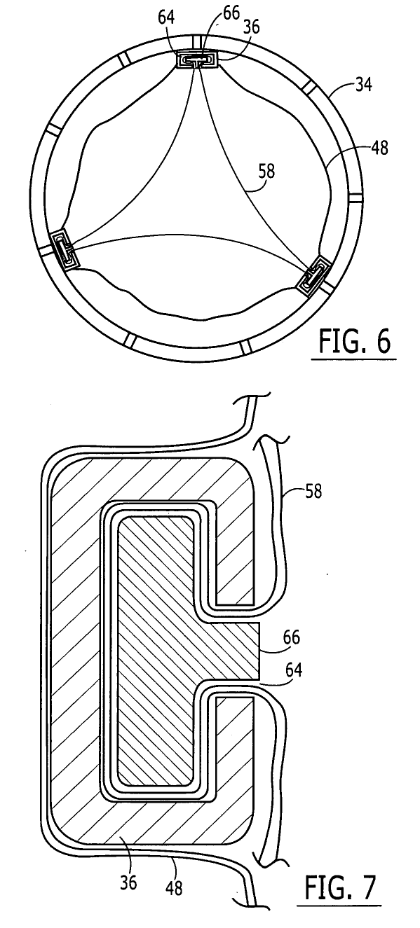

As illustrated in FIGS. 5A to 5D, the valve portion 38, illustrated without optional cloth 48, includes a tubular portion 58 and flaps 60. FIGS. 5A to 5D illustrate the tubular portion 58 in open and closed states. A portion of the tubular portion 58 is removed in FIGS. 5A and 5D to expose the flaps 60. The valve portion 38 is illustrated as having a tricuspid configuration but may also have a bicuspid configuration. Furthermore, flaps 60 are illustrated having a rectangular shape but may have any suitable size and configuration, e.g., triangular, etc. The specific number of flaps and the size and configuration chosen for the flaps 60 will depend on the size, configuration, and/or nature of the vessel in which the valve 32 will be implanted. Flaps 60 move from an opened position in which they extend substantially parallel with the tubular portion 58 and, thus allow blood flow along arrow 62A, as illustrated in FIGS. 5C and 5D, and a closed position, as illustrated in FIGS. 5A and 5B, in which the flaps 60 contact each other and, thus, prevent flow in one direction along arrow 62B across the valve portion 38. Valve portion 38 may be formed, for example, by invaginating a tubular structure, suturing the ends together at one or more suture points 62, and incising an inner wall of the invaginated tubular structure in at least two locations to form leaflets or flaps 60, which permit unidirectional blood flow.

Each of the flaps 60 may be constructed to form a pouch cavity, which fills with blood when the valve 32 is closed. This construction may minimize paravalvular leaks by a mechanism similar to a hyrdrofoil.

Anchor portion 34 may be a collapsible and radially re-expandable support, such as a stent, made from, for example, Nitinol, stainless steel, NP-35N alloy, etc. Anchor portion 34 may include markers, such as heavy metal markers, to facilitate placement within the body. Anchor portion 34 may include, for example, a gold, platinum, iridium tantalum or similar metal, etc., as a marker. The diameter of the anchor portion 34 may be, for example, between 4 mm and 50 mm. Anchor portion 34 may be cylindrical or may have a truncated conical form tapering toward the valve portion 38.

Anchor portion 12 is illustrated in FIG. 3 as having a sinusoid configuration but may have any type of cell design including, for example, zig-zag elements, ring members, braided strands, helically wound strands, consecutively attached ring members, tube members, a frame cut from solid tubes, etc. Further, the anchor portion 34 may be larger in diameter than the inner diameter of the vessel in which it will be implanted so as to facilitate maintenance of the valve 32 in the vessel.

Additional examples of suitable anchor portions for use with valve 32 include those described in U.S. Pat. No. 6,508,833 to Pavcnik et al., entitled “Multiple-sided Intraluminal Medical Device,” U.S. Pat. No. 6,464,720to Boatman et al., entitled “Radially Expandable Stent,” U.S. Pat. No. 6,231,598 to Berry et al., entitled “Radially Expandable Stent,” U.S. Pat. No. 6,299,635 to Frantzen, entitled “Radially Expandable Non-Axially Contracting Surgical Stent,” U.S. Pat. No. 4,580,568 to Gianturco, entitled “Percutaneous Endovascular Stent and Method for Insertion Thereof,” and U.S. Patent Application Publication No. 2001/0039450 to Pavcnik et al., entitled “Implantable Vascular Device,” each of which is expressly incorporated herein in its entirety by reference thereto.

A resorbable material may also be used for the anchor portion 34. A number of resorbable materials are believed to be conventional, and any suitable resorbable material may be used. Examples of suitable types of resorbable materials. include resorbable homopolymers, copolymers, blends of resorbable polymers, etc. Specific examples of suitable resorbable materials include poly-alpha hydroxy acids, such as polylactic acid, polylactide, polyglycolic acid (PGA), and polyglycolide, trimethylene carbonate, polycaprolactone, poly-beta hydroxy acids, such as polyhydroxybutyrate or polyhydroxyvalerate, and other polymers such as polyphosphazines, polyorganophosphazines, polyanhydrides, polyesteramides, polyorthoesters, polyethylene oxide, polyester-ethers (e.g., polydioxanone), polyamino acids (e.g., poly-L-glutamic acid or poly-L-lysine), etc. There are also a number of naturally derived resorbably polymers that may be suitable, including modified polysaccharides, such as cellulose, chitin, and dextran, and modified proteins, such as fibrin and casein, etc.

FIG. 6 is a cross-sectional view of valve 32 taken along line 6-6 in FIG. 3. As illustrated in FIG. 6, connectors 36 have a roughly C-shaped cross section with a slot 64.

The connectors 36 may be connected to the valve portion 38, for example, by suturing, stapling, riveting and chemical adhesion, etc. Connectors 36 may also be connected to the valve portion 38 mechanically, as illustrated in FIG. 7. FIG. 7 is a cross-sectional view taken along line 7-7 in FIG. 3. As illustrated in FIG. 7, a T-shaped member 66 is slid into slot 64 along with tubular portion 58 thereby securing connector 36 to valve portion 38 via tubular portion 58. T-shaped member 66 may be sized and shaped to provide a snug fit within slot 64. As indicated above, connector 36 may be connected to valve portion 38 using suturing, stapling, riveting, chemical adhesion, etc., in which case, the cross-section of the connector 36 may not need to have slot 64 and may have any other suitable shape.

Valve 32 or stent 24 (FIG. c) may be folded and radially compressed for insertion into sheath 10 using, for example, a crimping device including a plurality of adjustable plates resembling a typical single lens reflex (SLR) camera variable restrictor. Each plate moves along a line passing off an opening in the center, and all plates are equidistant from the center opening. The plates may be adapted to move simultaneously by a lever and transmission.

The placement of the valve 32 in the aorta (AO) may need to be precise in order to avoid blocking the opening to the coronary arteries, which branch off the aorta (AO). Separation of the anchor portion 34 and the valve portion 38 may allow for the use of a shorter valve portion and may facilitate placement of the valve portion 38 in the aorta (AO) without blocking the coronary arteries by the valve portion 38 or the anchor portion 34. In valves having stents disposed within or over the valve, the valves may need to be long enough to accommodate a stent of sufficient length to assure fixation and support of the valve. Separation of the valve and the stent may allow for the use of a shorter valve and, thus, may provide a surgeon more leeway in placement of the valve because the connectors 36 may be placed adjacent the opening of the coronary arteries without presenting any danger of blockage.

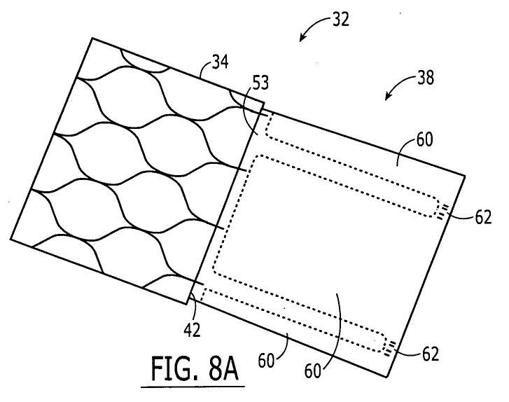

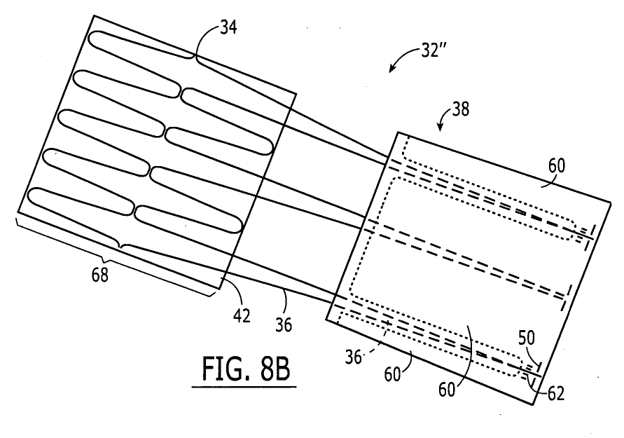

FIG. 8A illustrates a valve 32′ similar to that illustrated in FIG. 3 except that the valve portion 38 is directly connected on its distal end 53 to the proximal end 42 of the anchor portion 34 via, for example, sutures, staples, rivets, chemical adhesion, etc. Valve portion 38 is supported solely via its connection on its distal end 53 to the anchor portion 34 and is, in effect suspended by the anchor portion 34. As in the arrangement illustrated in FIG. 3, valve portion 38 does not have an additional stent disposed within or over tubular portion 58, which, as indicated above, may adversely affect the performance of the valve 32. That is, tubular portion 58 may be stentless. Alternatively, as indicated above, valve portion 38 may include a stent to maintain its expanded position. During insertion, the anchor portion 34 may be arranged within the distal portion 18 of the sheath 10, and the valve portion 38 may be arranged within the proximal portion 16 of the sheath 10.

The insertion method may also be used to implant the valve 32″ illustrated in FIG. 8B, which is similar to that illustrated in FIG. 3 except that the anchor portion 34 has a horizontal sinusoidal configuration and the connectors 36 are integral with the anchor portion 34. The anchor portion 34 has a main body portion 68 and connectors 36 that are integral with the anchor portion 34 and extend beyond a proximal end 42 of the main body portion 68. The valve portion 38 may be connected to a proximal portion of the connectors 36 such that a gap exists between the body portion 68 and the valve portion 38. The longer the gap, and the fewer the number of connectors 36, the less the expansion of the body portion 68 may affect the functioning of the valve portion 38. The above applies to the arrangements illustrated in FIGS. 3 and 8A as well. Further, with respect to the arrangement illustrated in FIG. 8B, the larger the number of sinusoids in the main body portion 68, the less the expansion of the body portion 68 may affect the functioning of the valve portion 38. During insertion, the valve portion 38 may be arranged within the proximal portion 16 of the sheath 10, and the anchor portion 38 may be arranged within the distal portion 18 of the sheath 10.

When the valves are used as a cardiac valve prosthesis in the aorta or main pulmonary artery, it is possible to mount the valve proximal to the native valve, within the native cardiac valve (with or without stenting of the native valve) or distal to the native valve, e.g., in the ascending aorta (AO), descending aorta or distal the main pulmonary artery. The valve may be used in place of the tricuspid valve, mitral valve and in artificial or biological conduits that may connect different chamber in the cardiovascular system, e.g., right ventricle (RV) to pulmonary artery conduits, intracardiac or extracardiac Fontan connections, left ventricle (LV) to ascending aorta (AO), etc.

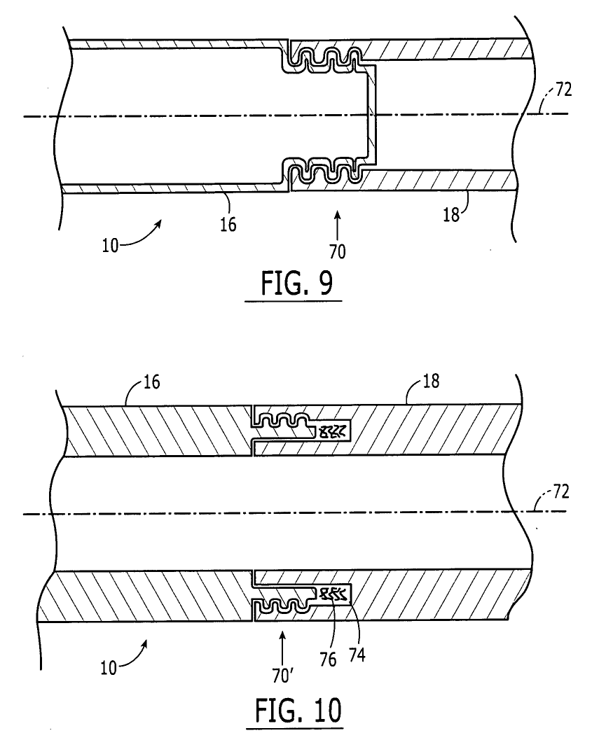

As indicated above, prior to shifting the proximal and distal portions 16, 18 apart to expose the medical device, the proximal and distal portions 16, 18 may be disconnected. A distal end of the proximal portion 16 and a proximal end of the distal portion 18 may be releasably connectable. For example, the proximal and distal portions 16, 18 may be connected via a threaded connection 70, as illustrated in FIG. 9. The sheath 10 may be separated into the proximal and distal portions 16, 18 by rotating these portions in opposite directions about a longitudinal axis 72 of the sheath 10.

A sheath 10 with a threaded connection 70″ is illustrated in FIG. 10. The sheath 10 may include a pocket 74 for delivery of a medical device or, drug 76 into the body of the patient. Pocket 74 is opened upon disconnection of the proximal portion 16 and the distal portion 18 of the sheath 10. Pocket 74 may be internally threaded to receive an end of the proximal portion 16, which may also be threaded.

The proximal and distal portions 16, 18 may be magnetically connected, as illustrated in FIG. 11. A coil 76 may be connected, for example, to an end of the proximal portion 16 and a magnetically-attractable member, such as a permanent magnet 78, for example, in the form of a ring, may be connected, for example, to an end of the distal portion 18. To secure the ends of the proximal and distal portions 16, 18 together, a current is passed through the coil 76 to generate a magnetic field which is attracted to the magnetic field produced by the permanent magnet 78. A controller 80 may be used to control the current supplied to coil 76 via line 82. The permanent magnet 78 may be replaced by a second coil and controller, such that both portions of the sheath 10 include an electromagnet. The coil 76 and line 82 are illustrated connected to an outer surface of the sheath 10, but they may also be connected to an inner surface of the sheath 10, embedded within the sheath 10, or extended through a lumen in a wall of the sheath 10.

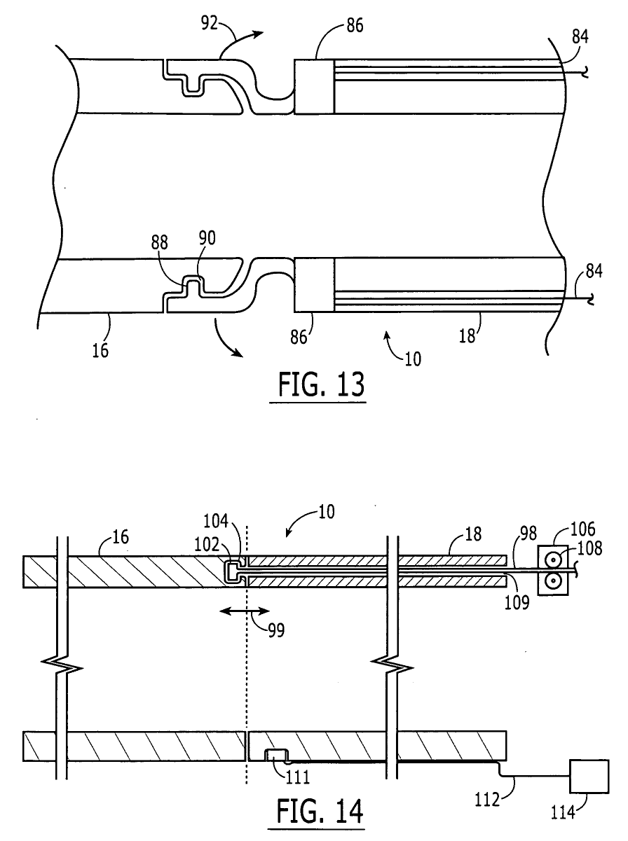

As illustrated in FIG. 13, line 84 may be connected to a motor or servo 86 used to control a latch 88. Latch 88 may move in the direction of arrow 92 between a connected position illustrated in FIG. 13, in which the latch 88 sits in a slot 90, and an unconnected position in which latch 88 is pivoted by motor or servo 86 out of slot 90. A controller connected to line 84 may be used to power the motor or servo 86 and, thus, open and close latch 88.

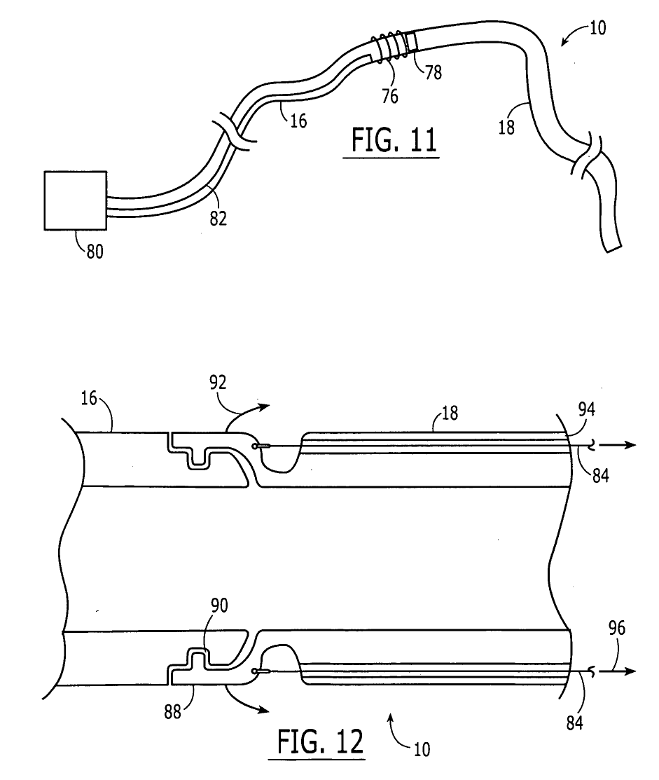

Line 84 may also be used to manually pivot the latch 88 between a locked and unlocked position. As illustrated in FIG. 12, line 84 may be slidingly disposed within lumen 94 and may connect at one end to latch 88. Pulling line 84 in a direction of arrow 96 may pivot latch 88 and disconnect the proximal and distal portions 16, 18 of sheath 10.

FIG. 14 illustrates a sheath 10 including a stylet 98 used to move the proximal and distal portions 16, 18 of the sheath 10 relative to each other. A head 102 of stylet 98 may be retained in a chamber 104 in proximal portion 16. Head may be rotatably and/or loosely secured in chamber 104 to allow it to rotate within chamber 104. Alternatively, head 102 may fit tightly within chamber 104 or maybe otherwise be fixed to proximal portion 16. For example, head 102 may be secured in a bearing secured to the proximal portion 16.

A mechanism 106, including, for example, powered wheels 108, may be used to shift stylet 98 within lumen 109 in distal portion 18 to shift the proximal and distal portions 16, 18 of sheath 10 toward and away from each other longitudinally, e.g., in direction of arrow 99, in a precise and controlled manner. Counter-rotation of wheels 108 in one direction, for example, shifts the proximal portion 16 away from the distal portion 18, and counter-rotation of wheels 108 in the opposite direction shifts the proximal and distal portions 16, 18 toward each other. The wheels 108 and the stylet 98, or at least the portion of the stylet 98 contactable by the wheels 108, may have a high degree of friction, e.g., a high coefficient of friction, to provide for precision and control and to reduce or avoid slippage between the wheels 108 and the stylet 98. The wheels 108 may include a toothing or gearing engageable with complementary toothing or gearing on the stylet 98. That is, wheels 108 and stylet 98 may have a rack-and-pinion arrangement. Any other mechanism for shifting a stylet or other elongate member may be used.

Further, separation of distal and proximal portions 16, 18 may be achieved by computerized control, e.g., by computerized control of mechanism 106. The mechanism 106 may include one or more encoder or sensor to determine the relative positioning or distal and proximal portions 16, 18. A scale or graduation may be provided on stylet 98 for indication of the relative movement or positioning of distal and proximal portions 16, 18.

The configuration illustrated in FIG. 14 may also be reversed, and stylet may extend through proximal portion 16 instead of distal portion 18. Sheath 10 may also include a visualization device 111, such as an ultrasound device, e.g., an ultrasound catheter, wire, camera, or transducer, which provides visualization of the vessel or organ structure and which may allow for exact placement of an implantable device carried by the sheath 10. Wire 112 connected, for example, to a surface of distal portion 18, may communicate the signals from the visualization device 111 to an image processor and/or a monitor 114. Wireless communication between the device 111 and an image processor and/or monitor 114 may also be provided.

Stylet 98 may be slidingly connected to an inside or outside surface of the sheath 10 rather than passing through lumen 109. Further, instead of wheels 108, mechanism 106 may include gears or other structure configured to engage corresponding gears or other structure on the stylet 98. For example, a ratchet and pawl arrangement may be provided for advancement of the stylet 98.

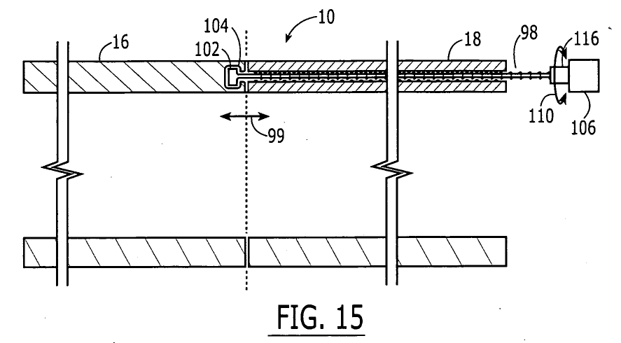

FIG. 15 illustrates a sheath 10 including a threaded connecting member 100 instead of stylet 98. Connecting member 100 is threaded within distal portion 18. Head 102 may be rotatably and/or loosely secured in chamber 104 to allow it to rotate within chamber 104 while connecting member 100 is threaded into and out of distal portion 18. The end of the connecting member 100 may be secured in a bearing secured to proximal portion 16.

One end of connecting member 100 is secured to mechanism 106 including, for example, a rotating element 116, which rotates connecting member 100 in a direction indicated by arrow 110. Rotation of connecting member 100 shifts this element further into and out of distal portion 18 causing the proximal portion 16 to shift away from and toward the distal portion 18 in the direction of arrow 99 in a precise and controlled manner. To enhance precision and control of the relative movement and position of distal and proximal portions 16, 18, the mechanism 106 may be computer controlled and may include one or more encoder or sensor to determine relative movement and positioning of distal and proximal portions.

While FIG. 15 illustrates a single threaded connecting member 100, it should be appreciated that other threaded or screw mechanisms may be provided for controlling relative movement and positioning of the distal and proximal portions 16, 18. For example, a differential screw mechanism may be provided, e.g., for providing both coarse and fine relative positioning and movement between the distal and proximal portions.

Alternatively, mechanism 106 may be dispensed with and the stylet 98 and connecting member 100 may be shifted/rotated manually. Indicators, e.g., a scale for the stylet or micrometer indicator for connecting member 100, may be provided for indicating relative positioning or movement between the distal and proximal portions 16, 18. Further, additional stylets 98 or connecting members 100 may be included in sheath 10. One or more guides may be provided, e.g., diametrically opposite to the stylet 98 or connecting member 98, to maintain longitudinal alignment between distal and proximal portions 16, 18.

Although explained in connection with cardiac heart valves implanted in the aortic position, the insertion methods described herein may be used to implant medical devices in other non-cardiac vessels or in other channels in the body, for example, in the veins, esophagus, stomach, ureter, bladder, urethra, biliary passes, lymphatic system, intestines, in CNS shunts and in the Fallopian tubes or other portions of the reproductive system, etc. The valve prosthesis may be used to replace a natural valve or to establish a new valve function in one of the channels in the body that does not naturally include a valve. The valve may be arranged to provide that fluids, such as blood, flows in only one direction through the valve. In persons having varicose veins, the blood flows in the wrong direction. A valve hereof may, for example, be placed in the varicose vein to prevent flow of blood in the wrong direction.

The foregoing description and example embodiments have been set forth for illustrative purposes only and are not intended to be limiting. Each of the disclosed aspects and example embodiments may be considered individually or in combination with other aspects, embodiments, and variations. Modifications of the described example embodiments may be made without departing from the spirit and scope hereof.

What is claimed is:

1. A system, comprising:
a) an insertion sheath sized for insertion of a medical device into a bodily vessel, the insertion sheath including proximal and distal portions that are releasably connectable to each other, the medical device deliverable into the bodily vessel one of (a) through and (b) in the insertion sheath and positionable within the sheath; and
b) an elongate member longitudinally movably connected along a longitudinal axis of the insertion sheath to a first one of the proximal portion and distal portion of the insertion sheath and secured to a second one of the proximal portion and distal portion, longitudinal movement of the elongate member along the longitudinal axis shifting the proximal and distal portions away from each other to expose the medical device.

2. The system according to claim 1, wherein the elongate member is screwed within the first one of the proximal portion and distal portion of the sheath and connected to the second one of the proximal portion and distal portion, the elongate member rotatable relative to the second one of the proximal portion and distal portion.

3. The system according to claim 1, wherein the elongate member has a head held and rotatable within a chamber in the second one of the proximal portion and distal portion of the sheath.

4. The system according to claim 1, further comprising a mechanism configured to shift the elongate member along the longitudinal axis.

5. The system according to claim 4, where the mechanism includes at least one wheel configured to roll on the elongate member to shift the elongate member along the longitudinal axis.

6. The system according to claim 4, wherein the mechanism is configured to rotate the elongate member.

7. The system according to claim 1, further comprising a visualization device connected to the sheath.

8. A system, comprising:
a) an implantable medical device;
b) an insertion sheath sized for insertion of the medical device into a bodily vessel, the insertion sheath including proximal and distal portions that are releasably connectable to each other, the medical device arranged within the insertion sheath and deliverable into the bodily vessel one of (a) through and (b) in the insertion sheath; and
c) an elongate member longitudinally movably connected along a longitudinal axis of the insertion sheath to a first one of the proximal portion and distal portion of the insertion sheath and secured to a second one of the proximal portion and distal portion, longitudinal movement of the elongate member along the longitudinal axis shifting the proximal and distal portions away from each other to expose the medical device.

9. The system according to claim 8, wherein the elongate member is slidingly connected along a longitudinal axis of the insertion sheath to a first one of the proximal portion and distal portion of the insertion sheath and secured to a second one of the proximal portion and distal portion.

10. The system according to claim 8, wherein the elongate member is screwed within the first one of the proximal portion and distal portion of the sheath and connected to the second one of the proximal portion and distal portion, the elongate member rotatable relative to the second one of the proximal portion and distal portion.

11. A system, comprising:
a) an insertion sheath sized for insertion of a medical device into a bodily vessel, the insertion sheath including proximal and distal portions that are releasably connectable to each other, the medical device deliverable into the bodily vessel one of (a) through and (b) in the insertion sheath and positionable within the sheath; and
b) means for shifting the proximal and distal portions away from each other to expose the medical device.

12. A system, comprising:
a) an implantable medical device;
b) an insertion sheath sized for insertion of the medical device into a bodily vessel, the insertion sheath including proximal and distal portions that are releasably connectable to each other, the medical device arranged within the insertion sheath and deliverable into the bodily vessel one of (a) through and (b) in the insertion sheath; and
c) means for shifting the proximal and distal portions away from each other to expose the medical device.
